(19) United States
(12) Patent Application Publication
(10) Pub. No.: US 2016/0237012 A1
(43) Pub. Date: Aug. 18, 2016

(54) ANTICANCER AGENTS AND PROCESS OF MAKING THEREOF

(71) Applicant: Golden Biotechnology Corporation, Jersey City, NJ (US)

(72) Inventors: Sheng-Yung LIU, New Taipei City (TW); Chih-Ming Chen, New Taipei City (TW)

(21) Appl. No.: 15/045,259

(22) Filed: Feb. 17, 2016

Related U.S. Application Data
(60) Provisional application No. 62/117,408, filed on Feb. 17, 2015.

(51) Int. Cl. C07C 39/19; C07C 37/14; C07D 307/33; C07C 69/96; C07C 68/00; C07C 49/743; C07C 57/42

(52) U.S. Cl. CPC C07C 39/19; C07C 49/743; C07C 37/14; C07C 57/42; C07C 69/96; C07C 68/00; C07D 307/33

(57) ABSTRACT

Provided herein are compositions and processes of making of anticancer compounds useful for cancer treatments. These cyclohexenone compounds show an unexpected result against certain cancer cells compared to their known analogs.

### BACKGROUND OF THE INVENTION

The present disclosure relates to novel anticancer agents, and processes of making thereof.

### SUMMARY OF THE INVENTION

In one aspect, there are provided a compound of formula I:

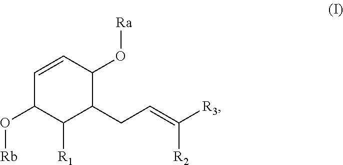

or a pharmaceutically acceptable salt, metabolite, solvate or prodrug thereof, wherein each of Ra and Rb is hydrogen, C(═O)OR₅, C(═O)R₅, C(═O)NR₅R₆, or C₁-C₁₂alkyl,
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

In one aspect, there are provided a compound of formula II:

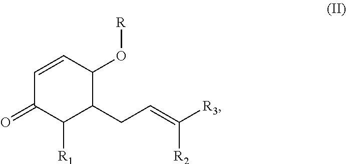

or a pharmaceutically acceptable salt, metabolite, solvate or prodrug thereof, wherein R is hydrogen, C(═O)OR₅, C(═O)R₅, C(═O)NR₅R₆, or C₁-C₁₂alkyl, R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

In one aspect, there are provided a compound of formula III:

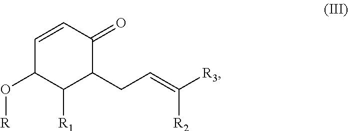

or a pharmaceutically acceptable salt, metabolite, solvate or prodrug thereof, wherein R is hydrogen, C(═O)OR₅, C(═O)R₅, C(═O)NR₅R₆, or C₁-C₁₂alkyl, R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

In one aspect, there are provided a compound of formula IV:

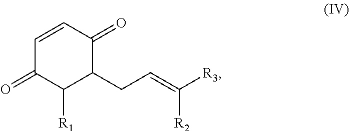

or a pharmaceutically acceptable salt, metabolite, solvate or prodrug thereof, wherein
R₁ is C₁-C₁₂alkyl, NR₅R₆, OR₅, SR₅, or halogen;
each of R₂ and R₃ independently is hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

In one aspect, there are provided a compound of formula V:

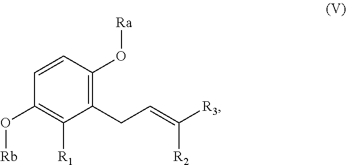

or a pharmaceutically acceptable salt, metabolite, solvate or prodrug thereof, wherein each of Ra and Rb is hydrogen, C(═O)OR₅, C(═O)R₅, C(═O)NR₅R₆, or C₁-C₁₂alkyl
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

In another aspect of the present invention, there are provided processes for preparing a compound of formula VI:

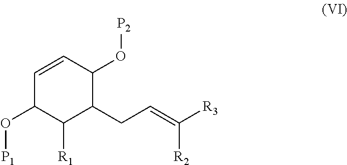

comprising a step of reacting a compound of formula II,

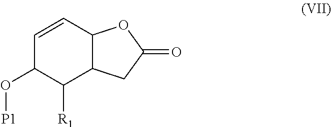

with a compound (VIII), Ph₃PCHR₂R₃L (VIII), in the presence of a reducing agent, and a base,
wherein L is a leaving group, each of P₁ and P₂ is a hydroxyl protecting group or R;
R is hydrogen, C(═O)OR₅, C(═O)R₅, C(═O)NR₅R₆, or C₁-C₁₂alkyl;
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

In another aspect of the present invention, there are provided processes for preparing a compound of formula IX:

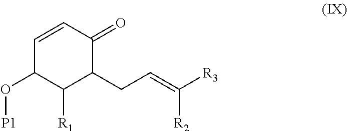

comprising reacting an enol or enolate compound of formula X,

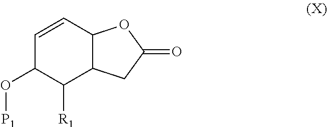

with a compound (XI),

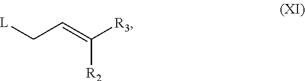

under suitable conditions, wherein
wherein L is a leaving group, P₁ is a hydroxyl protecting group or R; R is a hydrogen, C(═O)OR₅, C(═O)R₅, C(═O)NR₅R₆, or a C₁-C₁₂alkyl;
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The present invention provides novel anticancer agents and processes of making thereof. For example, the following exemplary anticancer compounds 1-6 are prepared and test for anticancer activities over e.g., liver cancer cells and breast cancer cells.

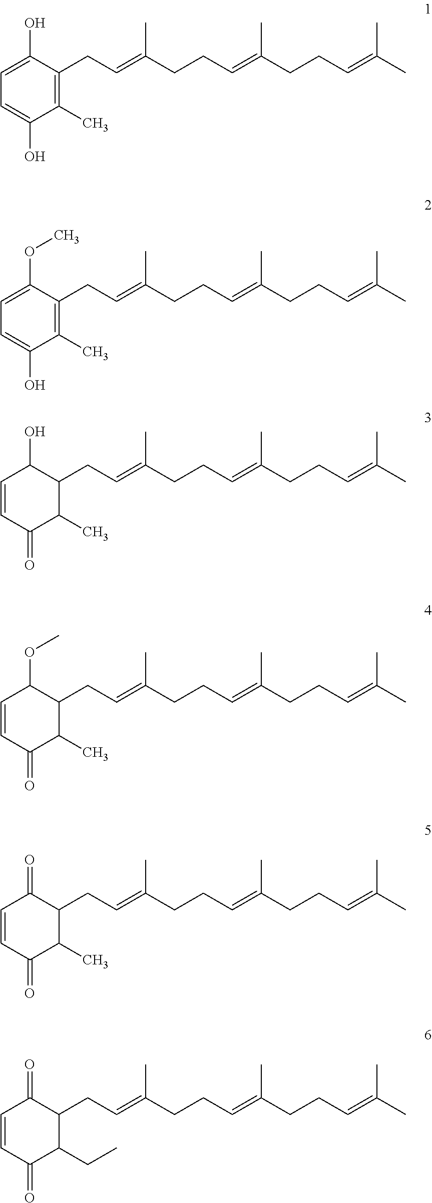 [SMILES: CC(C)=CCC/C(C)=C/CC/C(C)=C/Cc1c(O)ccc(O)c1C ; COc1ccc(O)c(C)c1C/C=C(\C)CC/C=C(\C)CCC=C(C)C ; CC(C)=CCC/C(C)=C/CC/C(C)=C/CC1C(O)C=CC(=O)C1C ; COC1C=CC(=O)C(C)C1C/C=C(\C)CC/C=C(\C)CCC=C(C)C ; CC(C)=CCC/C(C)=C/CC/C(C)=C/CC1C(=O)C=CC(=O)C1C ; CCC1C(=O)C=CC(=O)C1C/C=C(\C)CC/C=C(\C)CCC=C(C)C]

In some embodiments, there are provided herein a compound of formula I

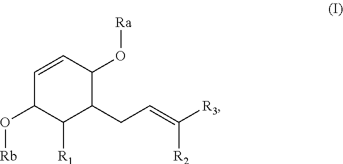

or a pharmaceutically acceptable salt, metabolite, solvate or prodrug thereof, wherein each of Ra and Rb is a hydrogen, C(═O)OR₅, C(═O)R₅, C(═O)NR₅R₆, or a C₁-C₁₂alkyl,
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11. In certain embodiments, R is a hydrogen, C(═O)C₃H₈, C(═O)C₂H₅, or C(═O)CH₃. In certain embodiments, each of R₁, R₂ and R₃ independently is hydrogen, methyl, ethyl, propyl, butyl, pentyl, hexyl, or heptyl. In certain embodiments, each of R₂ and R₃ independently is (CH₂CH═C(CH₃)(CH₂))ₘ—R₄. In certain embodiments, m=2. In certain embodiments, R₄ is H, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In certain embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂CH═C(CH₃)₂.

In some embodiments, there are provided herein a compound of formula II

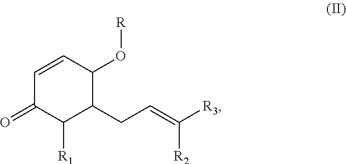

or a pharmaceutically acceptable salt, metabolite, solvate or prodrug thereof, wherein R is a hydrogen, C(═O)OR₅, C(═O)R₅, C(═O)NR₅R₆, or a C₁-C₁₂alkyl, R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;

m=0-11. In certain embodiments, R is a hydrogen, C(═O)C₃H₈, C(═O)C₂H₅, or C(═O)CH₃. In certain embodiments, each of R₁, R₂ and R₃ independently is hydrogen, methyl, ethyl, propyl, butyl, pentyl, hexyl, or heptyl. In certain embodiments, each of R₂ and R₃ independently is (CH₂CH═C(CH₃)(CH₂))ₘ—R₄. In certain embodiments, m=2. In certain embodiments, R₄ is H, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In certain embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂CH═C(CH₃)₂. In certain embodiments, the compound is

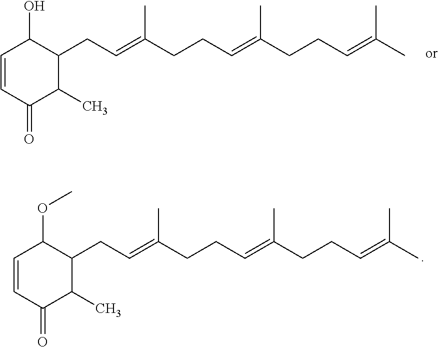 [SMILES: CC(C)=CCC/C(C)=C/CC/C(C)=C/CC1C(O)C=CC(=O)C1C ; COC1C=CC(=O)C(C)C1C/C=C(\C)CC/C=C(\C)CCC=C(C)C]

In some embodiments, there are provided herein a compound of formula III

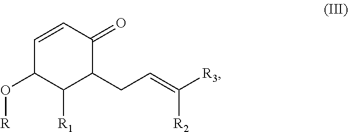

or a pharmaceutically acceptable salt, metabolite, solvate or prodrug thereof, wherein R is a hydrogen, C(═O)OR₅, C(═O)R₅, C(═O)NR₅R₆, or a C₁-C₁₂alkyl, R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;

m=0-11. In certain embodiments, R is a hydrogen, C(═O)C₃H₈, C(═O)C₂H₅, or C(═O)CH₃. In certain embodiments, each of R₁, R₂ and R₃ independently is hydrogen, methyl, ethyl, propyl, butyl, pentyl, hexyl, or heptyl. In certain embodiments, each of R₂ and R₃ independently is (CH₂CH═C(CH₃)(CH₂))ₘ—R₄. In certain embodiments, m=2. In certain embodiments, R₄ is H, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In certain embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂CH═C(CH₃)₂.

In some embodiments, there are provided herein a compound of formula IV

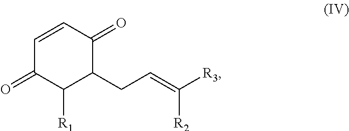

or a pharmaceutically acceptable salt, metabolite, solvate or prodrug thereof, wherein
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;

m=0-11. In certain embodiments, each of R₁, R₂ and R₃ independently is hydrogen, methyl, ethyl, propyl, butyl, pentyl, hexyl, or heptyl. In certain embodiments, each of R₂ and R₃ independently is (CH₂CH═C(CH₃)(CH₂))ₘ—R₄. In certain embodiments, m=2. In certain embodiments, R₄ is H, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In certain embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂CH═C(CH₃)₂.

In some embodiments, there are provided herein a compound of formula V

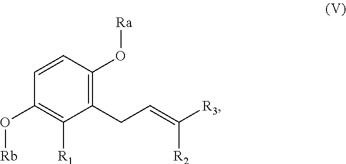

or a pharmaceutically acceptable salt, metabolite, solvate or prodrug thereof, wherein each of Ra and Rb is a hydrogen, C(═O)OR₅, C(═O)R₅, C(═O)NR₅R₆, or a C₁-C₁₂alkyl
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

In certain embodiments, each of R₁, R₂ and R₃ independently is hydrogen, methyl, ethyl, propyl, butyl, pentyl, hexyl, or heptyl. In certain embodiments, each of R₂ and R₃ independently is (CH₂CH═C(CH₃)(CH₂))ₘ—R₄. In certain embodiments, m=2. In certain embodiments, R₄ is H, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In certain embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂CH═C(CH₃)₂. In certain embodiments, the compound is

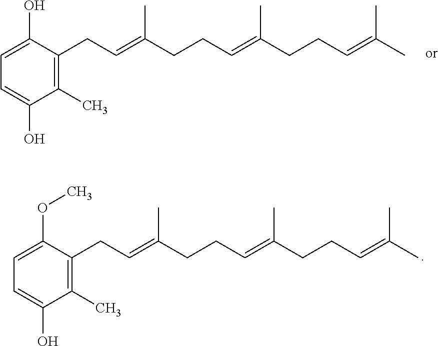 [SMILES: CC(C)=CCC/C(C)=C/CC/C(C)=C/Cc1c(O)ccc(O)c1C ; COc1ccc(O)c(C)c1C/C=C(\C)CC/C=C(\C)CCC=C(C)C]

The following are some non-limited examples of invention compounds useful for anticancer treatments:

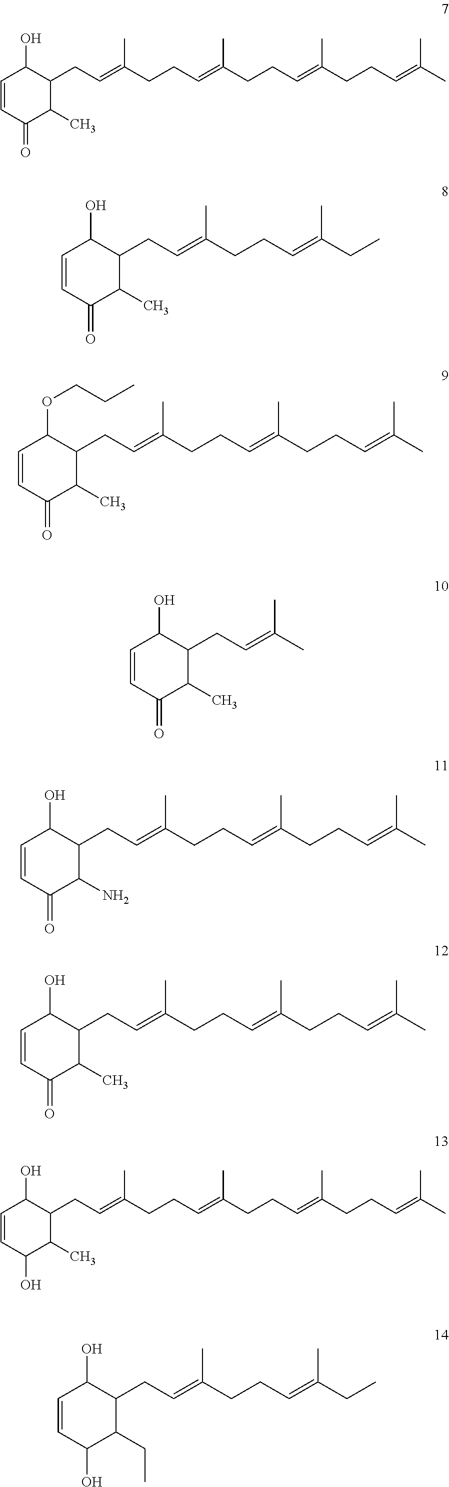

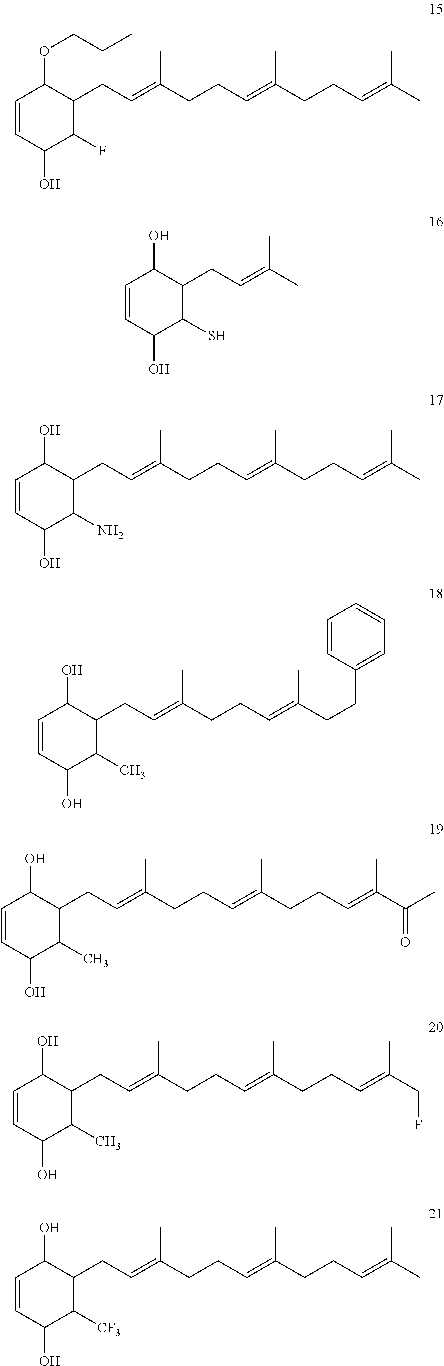

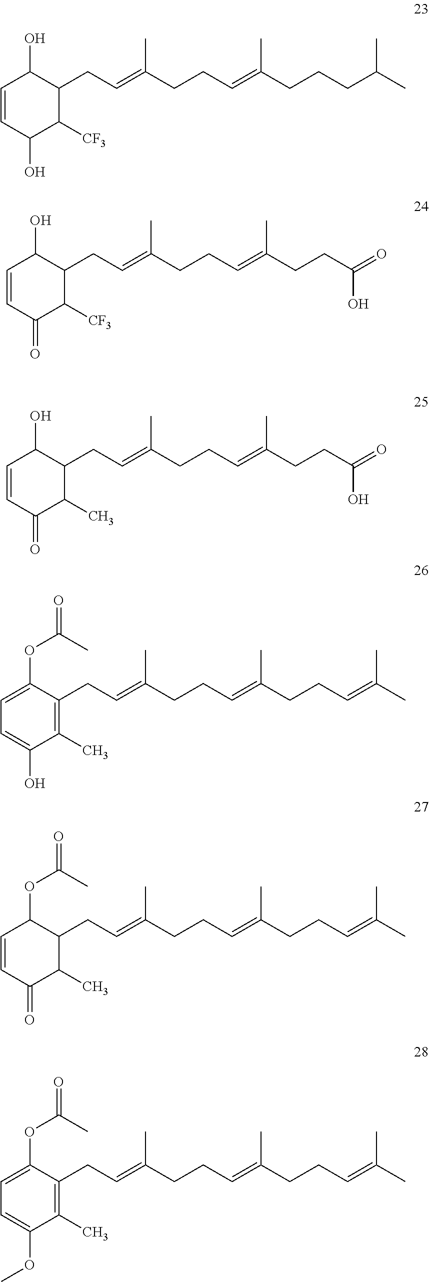 [SMILES: C/C(=C\CC1C(O)C=CC(O)C1C(F)(F)F)CC/C=C(\C)CCCC(C)C ; C/C(=C\CC1C(O)C=CC(=O)C1C(F)(F)F)CC/C=C(\C)CCC(=O)O ; C/C(=C\CC1C(O)C=CC(=O)C1C)CC/C=C(\C)CCC(=O)O ; CC(=O)Oc1ccc(O)c(C)c1C/C=C(\C)CC/C=C(\C)CCC=C(C)C ; CC(=O)OC1C=CC(=O)C(C)C1C/C=C(\C)CC/C=C(\C)CCC=C(C)C ; COc1ccc(OC(C)=O)c(C/C=C(\C)CC/C=C(\C)CCC=C(C)C)c1C]

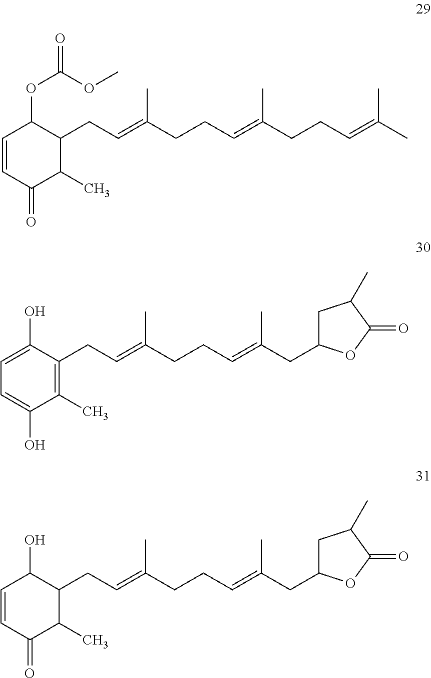 [SMILES: COC(=O)OC1C=CC(=O)C(C)C1C/C=C(\C)CC/C=C(\C)CCC=C(C)C ; C/C(=C\Cc1c(O)ccc(O)c1C)CC/C=C(\C)CC1CC(C)C(=O)O1 ; C/C(=C\CC1C(O)C=CC(=O)C1C)CC/C=C(\C)CC1CC(C)C(=O)O1]

In accordance with the present invention, there are provided processes for preparing a compound of formula VI:

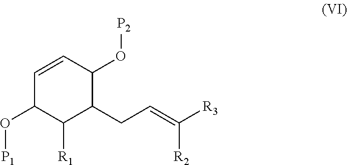

comprising a step of reacting a compound of formula II,

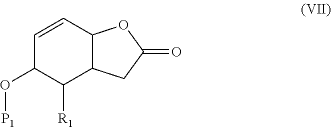

with a compound (VIII), Ph₃PCHR₂R₃L (VIII), in the presence of a reducing agent, and a base,
wherein L is a leaving group, each of P₁ and P₂ is a hydroxyl protecting group or R;
R is a hydrogen, C(═O)OR₅, C(═O)R₅, C(═O)NR₅R₆, or a C₁-C₁₂alkyl;
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

The reaction between a compound of formula (VII) and a compound of formula (VIII) is known as Wittig reaction. Since aldehydes, in general, are not chemically stable, the resulting aldehydes of compounds of formula (VII) after reduction, in some embodiments, are prepared in situ. Scheme I provides a non-limited exemplary route to prepare a compound of formulae (I) to (VI). Protection of the free hydroxyl group of Compound 35 follows by reduction of the lactone ring to afford the aldehyde from a compound of formula (VII), which then undergo Wittig reaction with Ph₃PCHR₂R₃I to prepare intermediate A. After deprotection of protecting group P1, Compound B is prepared. Compound B can then go through different reaction to afford invention compounds. For example, oxidation of Compound B gives Compounds C which can undergo deportation and optional hydroxyl group derivatization to afford Compound 36 of formula (II).

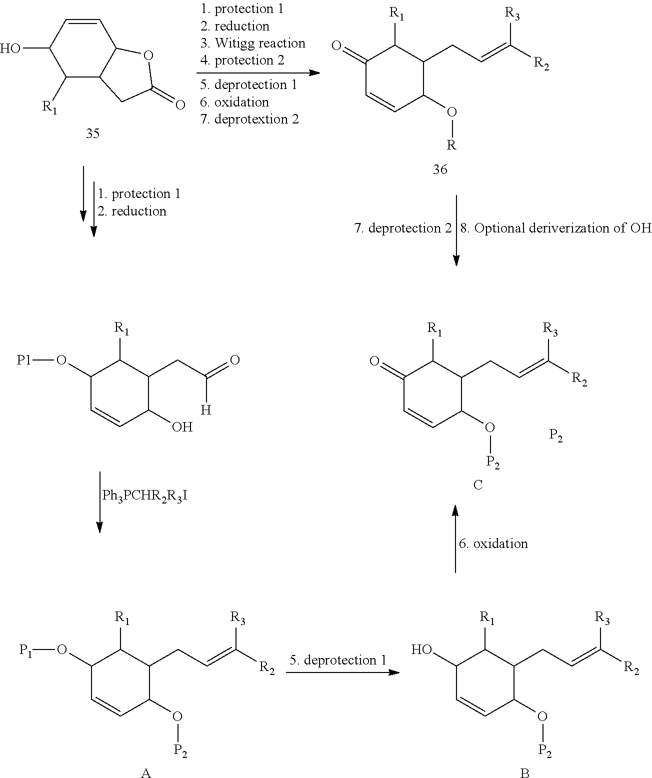

Scheme I. Exemplary Synthetic Scheme to Prepare an Exemplary Invention Compounds

Deprotection of Compound A follows by different degree of oxidation affords various of compounds which can derivatize to invention compounds of Formulae (II) to (V). Selective deprotection, and then derivatization afford compounds of formula (I). Selective deprotection, oxidation and then derivatization afford compounds of formulae (II) to (V). Under a more controlled setting, invention compounds can be prepared as shown in Scheme II.

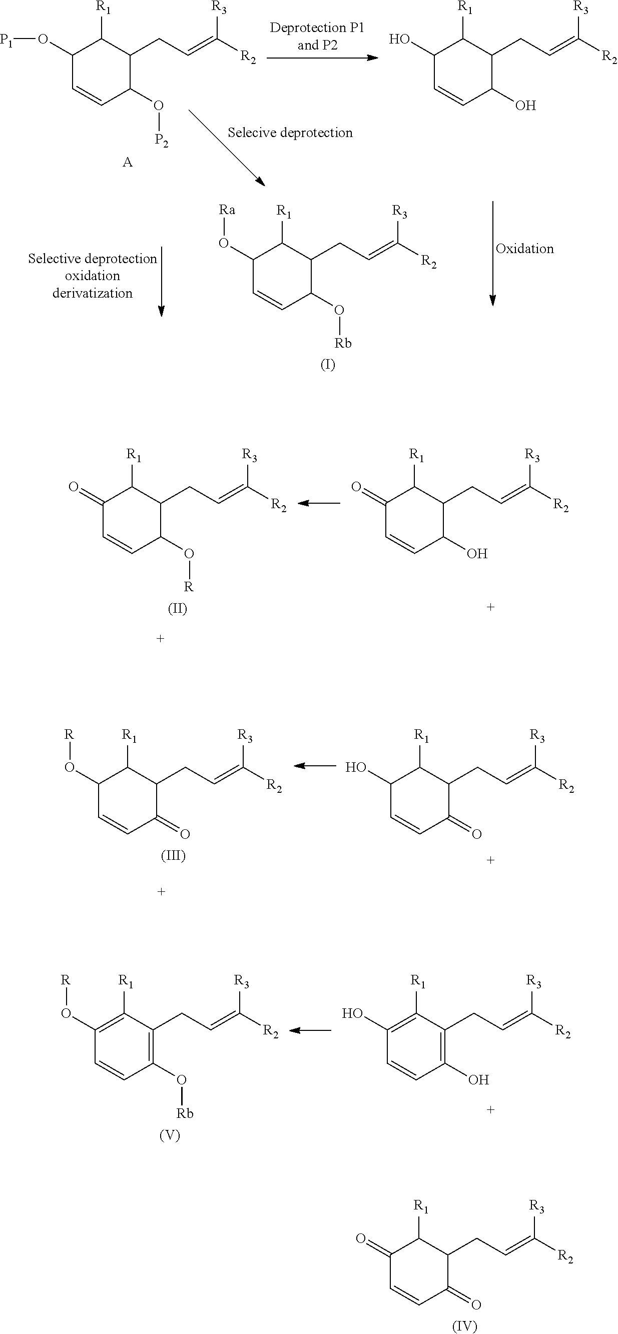

Scheme II: Exemplary Reactions to Prepare Invention Compounds by Selective Deprotection, Oxidation and Derivatization.

An aldehyde can be prepared from reduction of acylsilanes, carboxylic acids, acid halides, anhydride, esters, lactones, amides, nitriles, or the like. In some instances, an aldehyde can be prepared from oxidation of a free hydroxyl group. A skilled person in the art can readily consider other suitable reaction based on this invention to prepare the aldehyde of a compound of formula (VII). In some embodiments, the aldehyde of a compound of formula (VII),

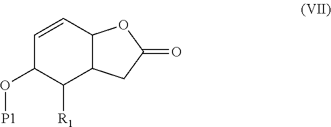

is prepared from reduction of a compound having the structure of

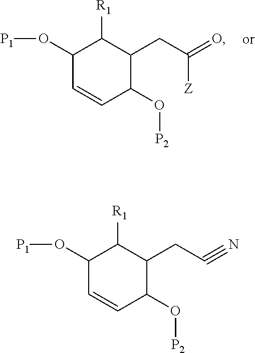

wherein Z is halogen, OR₅OC(═O)R₇, or NR₅R₆.

In some embodiments, the aldehyde of a compound of formula (VII),

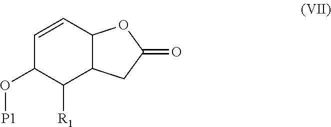

is prepared from oxidation of a compound having the structure of

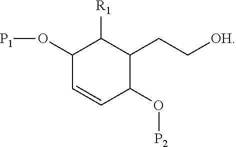

In some embodiments, P1 or P2 is any suitable hydroxyl protecting group that can survive Wittig reaction conditions. For example, P1 or P2 is C(═O)OR₅, C(═O)R₅, C(═O)NR₅R₆, C(═O)SR₅, C(═S)R₅, C(═S)NR₅R₆, or the like.

In some embodiments, said base is a base that can form an ylide from a compound of formula (III), for example, n-butyllithium (n-BuLi), or the like.

The Wittig reaction provided herein is applicable to many isoprene unit precursors. For example, the reaction is applicable where R₂ is CH₃ and R₃ is CH₂ substituted with (CH₂CH═C(CH₃)(CH₂))ₘ—R₄, wherein is R₄ is hydrogen NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl; each of R₅ and R₆ is independently H or C₁-C₈alkyl; and R₇ is a C₁-C₈alkyl, OR₅ or NR₅R₆.

For example, without limitation, a skilled artisan may use the following isoprene precursors where P1 is a hydroxy protecting group.

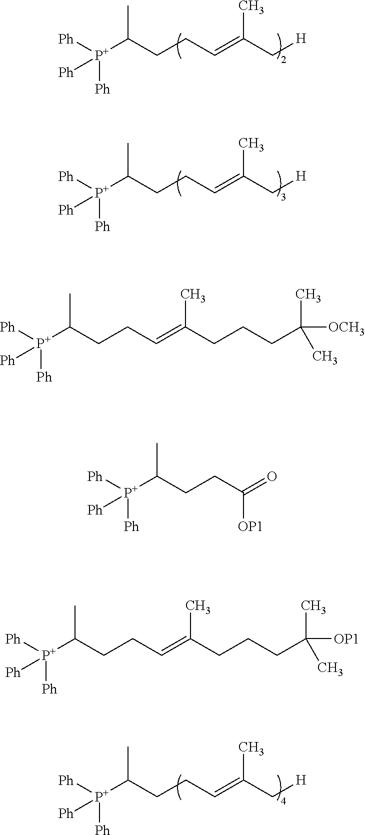

In certain embodiments, R₁ is H, methyl, ethyl, propyl, butyl, pentyl, or the like. In certain embodiments, R, R₁Ra, Rb independently is H, methyl, ethyl, propyl, butyl, pentyl, or the like. In certain embodiments, R or Rₐ is H, methyl, ethyl, propyl, butyl, pentyl, or the like. In certain embodiments, R or R_{b} is H, methyl, ethyl, propyl, butyl, pentyl, or the like.

In some embodiments, there are provided processes for preparing a compound of formula IX:

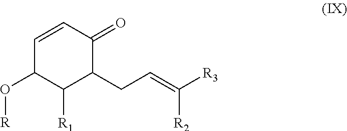

comprising reacting an enol or enolate compound of formula X,

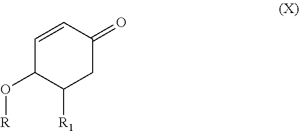

with a compound of formula (XI),

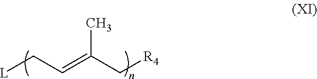

under suitable conditions, wherein
wherein L is a leaving group, R is a hydroxyl protecting group;
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

In some embodiments, the enol compound of formula X,

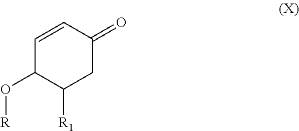

is prepared under suitable conditions (e.g., acid promotion or silyl trapping).

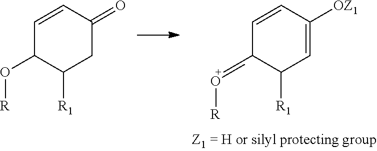

In some embodiments, the enolate compound of formula X, is prepared by reacting a compound of formula X with a strong base. A skilled artisan will readily find other suitable conditions follows the known procedure to prepare the enol or enolate compound of formula X.

In some embodiments, L is a leaving group that undergoes either SN1, SN2 or SNi reaction under suitable conditions. For example, L is a halogen such as Cl, Br or I. In some instances, L is hydroxyl derived leaving group such as a tosylate or methylate. Other suitable leaving groups may be used by a skilled artisan follows the readily available known procedure.

In certain embodiments, R₁ is H, methyl, ethyl, propyl, butyl, pentyl, or the like. In certain embodiments, R, R₁Rₐ, R_{b} independently is H, methyl, ethyl, propyl, butyl, pentyl, or the like. In certain embodiments, R or Rₐ is H, methyl, ethyl, propyl, butyl, pentyl, or the like. In certain embodiments, R or R_{b} is H, methyl, ethyl, propyl, butyl, pentyl, or the like.

Formation of Covalent Linkages by Reaction of an Electrophile with a Nucleophile

In certain embodiments, the compounds described herein are modified using various electrophiles or nucleophiles to form new functional groups or substituents. Table 1 entitled “Examples of Covalent Linkages and Precursors Thereof” lists selected, non-limiting examples of covalent linkages and precursor functional groups that are used to prepare the modified compounds. Precursor functional groups are shown as electrophilic groups and nucleophilic groups.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it is necessary in certain embodiments to protect reactive functional groups, for example hydroxy, amino, thiol or carboxy groups, where these are desired in the final product, to avoid their unwanted participation in the reactions. Protecting groups are used to block some or all reactive moieties and prevent such groups from participating in chemical reactions until the protective group is removed. In one embodiment, each protective group is removable by a different means. Protective groups that are cleaved under totally disparate reaction conditions fulfill the requirement of differential removal. In some embodiments, protective groups are removed by acid, base, and/or hydrogenolysis. Groups such as trityl, dimethoxytrityl, acetal and t-butyldimethylsilyl are acid labile and are used in certain embodiments to protect carboxy and hydroxy reactive moieties in the presence of amino groups protected with Cbz groups, which are removable by hydrogenolysis, and/or Fmoc groups, which are base labile. In other embodiments, carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In another embodiment, carboxylic acid and hydroxy reactive moieties are blocked with hydrolytically removable protective groups such as the benzyl group, while amine groups capable of hydrogen bonding with acids are blocked with base labile groups such as Fmoc. In another embodiment, carboxylic acid reactive moieties are protected by conversion to simple ester compounds as exemplified herein, or they are, in yet another embodiment, blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are optionally subsequently removed by metal or pi-acid catalysts. For example, an allyl-blocked carboxylic acid is optionally deprotected with a Pd(0)-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and cannot react. Once released from the resin, the functional group is available to react.

Typically blocking/protecting groups are, by way of example only:

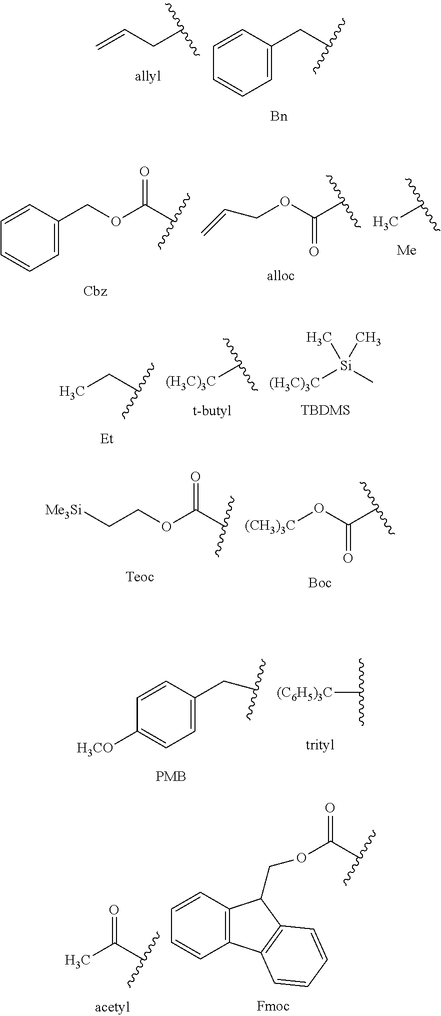

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound provided herein with acids. Pharmaceutically acceptable salts are also obtained by reacting a compound provided herein with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methyl amine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

The term “leaving group” as used herein may be any group which is usually known as a leaving group in organic synthesis, without limitation, for example: halogens such as fluorine, chlorine, bromine and iodine, alkylsulfonyloxy groups such as methanesulfonyloxy, trifluoromethanesulfonyloxy and ethanesulfonyloxy, arylsulfonyloxy groups such as benzenesulfonyloxy and p-toluenesulfonyloxy. Preferred “leaving groups” are halogens such as fluorine, chlorine, bromine and iodine.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

Unless defined otherwise, all technical and scientific terms used herein have the standard meaning pertaining to the claimed subject matter belongs. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless otherwise indicated, conventional methods of mass spectroscopy, NMR, HPLC, protein chemistry, biochemistry, recombinant DNA techniques and pharmacology are employed. Unless specific definitions are provided, the standard nomenclature employed in connection with, and the standard laboratory procedures and techniques of, analytical chemistry, synthetic organic chemistry, and medicinal and pharmaceutical chemistry are employed. In certain instances, standard techniques are used for chemical syntheses, chemical analyses, pharmaceutical preparation, formulation, and delivery, and treatment of patients. In certain embodiments, standard techniques are used for recombinant DNA, oligonucleotide synthesis, and tissue culture and transformation (e.g., electroporation, lipofection). In some embodiments, reactions and purification techniques are performed e.g., using kits of manufacturer's specifications or as commonly accomplished or as described herein.

As used throughout this application and the appended claims, the following terms have the following meanings:

The term “alkyl” as used herein, means a straight, branched chain, or cyclic (in this case, it would also be known as “cycloalkyl”) hydrocarbon containing from 1-10 carbon atoms. Illustrative examples of alkyl include, but are not limited to, methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl.

The term “C₁-C₈-alkyl” as used herein, means a straight, branched chain, or cyclic (in this case, it would also be known as “cycloalkyl”) hydrocarbon containing from 1-8 carbon atoms. Representative examples of alkyl include, but are not limited to, methyl, ethyl, n-propyl, iso-propyl, cyclopyl, n-butyl, sec-butyl, tert-butyl, cyclobutyl, n-pentyl, isopentyl, neopentyl, cyclopentyl, and n-hexyl.

The term “thioalkyl” as used herein, means an alkyl group, as defined herein, appended to the parent molecular moiety through a sulfur atom. Illustrative examples of thioalkyl include, but are not limited to, methylthio, ethylthio, butylthio, tert-butylthio, and hexylthio.

The term “halo” or “halogen” as used herein, means a —Cl, —Br, —I or —F.

As used herein, the term “sulfinyl” refers to a —S(═O)—R, where R is selected from the group consisting of alkyl, cycloalkyl, aryl, heteroaryl (bonded through a ring carbon) and heterocycloalkyl (bonded through a ring carbon).

As used herein, the term “sulfonyl” refers to a —S(═O)₂—R, where R is selected from the group consisting of alkyl, cycloalkyl, aryl, heteroaryl (bonded through a ring carbon) and heterocycloalkyl (bonded through a ring carbon).

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, nitro, haloalkyl, fluoroalkyl, fluoroalkoxy, and amino, including mono- and di-substituted amino groups, and the protected derivatives thereof. By way of example an optional substituents may be halide, —CN, —NO₂, or LₛRₛ, wherein each Lₛ is independently selected from a bond, —O—, —C(═O)—, —C(═O)O—, —S—, —S(═O)—, —S(═O)₂—, —NH—, —NHC(═O)—, —C(═O)NH—, S(═O)₂NH—, —NHS(═O)₂, —OC(═O)NH—, —NHC(═O)O—, or —(C₁-C₆ alkylene)-; and each Rₛ is selected from H, alkyl, fluoroalkyl, heteroalkyl, cycloalkyl, aryl, heteroaryl, or heterocycloalkyl. The protecting groups that may form the protective derivatives of the above substituents may be found in sources such as Greene and Wuts, above. In some embodiments, optional substituents are selected from halogen, —CN, —NH₂, —OH, —N(CH₃)₂, alkyl, fluoroalkyl, heteroalkyl, cycloalkyl, heterocycloalkyl, aryl, heteroaryl, alkoxy, aryloxy, alkylthio, arylthio, alkylsulfoxide, arylsulfoxide, alkylsulfone, and arylsulfone. In some embodiments, an optional substituents is halogen, —CN, —NH₂, —OH, —NH(CH₃), —N(CH₃)₂, alkyl, fluoroalkyl, heteroalkyl, alkoxy, fluoroalkoxy, —S-alkyl, or —S(═O)₂alkyl. In some embodiments, an optional substituent is selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, substituted groups are substituted with one of the preceding groups. In some embodiments, an optional substituent on an aliphatic carbon atom (acyclic or cyclic, saturated or unsaturated carbon atoms, excluding aromatic carbon atoms) includes oxo (═O).

The term “protected amine” refers to an amine with a removable protecting group which modifies the reactivity of an amine, against undesirable reaction during synthetic procedures and to be later removed. Examples of amine protecting groups include, but are not limited to, tert-butoxycarbonyl (Boc), 9-fluorenylmethyl carbonyl (Fmoc), triphenylmethyl (Tr) and carbobenzyloxy (Cbz). For example, to protect and activate the pyrimidine ring system with the 6-amino moiety in accordance with the present invention, bis-BOC, or bis-FMOC, CBZ, alloc, Teoc, methyl/ethyl-oxycarbonyl, bis-acetyl, or N-succinyl or N-phthaloyl may be used in addition to their mono-N protected analogs.

### EXAMPLE

### Example 1

### Preparation of Exemplary Anticancer Agent Core

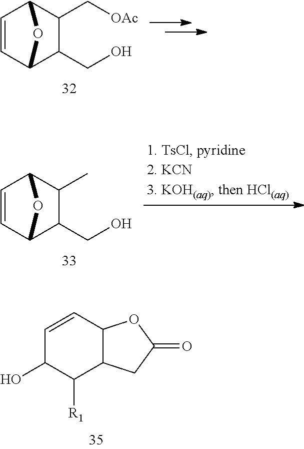

Compound 33 was prepared by a known method (e.g, J. Org. Chem. 2004, 69, 8789-8795) from compound 32. The exemplary intermediate 35a (R1=methyl) was prepared by the following steps.

### Step 1. Preparation of Compound 2-1

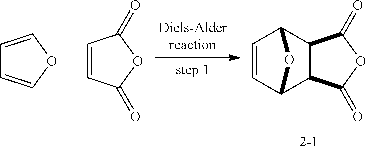

Dimethoxyl furan (4.67 g, 36.4 mmol) and diethyl fumarate (6.0 mL, 36.6 mmol) were in ethyl acetate (10 mL). The reaction mixture was stirred overnight at room temperature. Solvent was removed in vacuo and the residue was purified by column chromatography on silica gel (EtOAc/hexane 1:5) to yield 9.0 g of 2-1 (30.0 mmol, 83%); R_{f}=0.47 (EtOAc/hexane 1:3)

### Step 2. Preparation of Compound 2-2

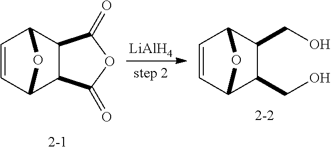

The solution of 2-1 (1.8 g, 6.0 mmol) in THF (12 mL) was added to the suspension of LiAlH₄ (455 mg, 12.0 mmol) in dry THF (12 mL) at ice bath under N₂. The reaction mixture was allowed to warm to room temperature. After stirring for 16 h, the reaction mixture was cooled to 0° C., and quenched carefully by sat. Na₂CO_{3(aq)} (1.5 mL) and H₂O (1.5 mL), stirred for another 1 h, filtered and concentrated to yield crude 2-2 (1.80 g); R_{f}=0.33 (EA).

### Step 3. Preparation Compound 2-3

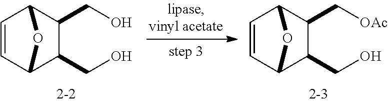 [SMILES: OC[C@@H]1[C@H](CO)[C@H]2C=C[C@@H]1O2 ; CC(=O)OC[C@H]1[C@@H](CO)[C@@H]2C=C[C@H]1O2]

Lipase PS (Amano) was added to a solution of crude 2-2 (4.5 g, 20.8 mmol) in vinyl acetate (57.5 mL). The reaction mixture was stirred at room temperature for 16 h, filtered to remove lipase PS. The filtrate was concentrated and the residue was purified by column chromatography on silica gel (EtOAc/hexane 1:1, then EA, R_{f}=0.41) to yield 2.5 g of 2-3 (9.7 mmol, 47%).

### Step 4. Preparation of Compound 2-4

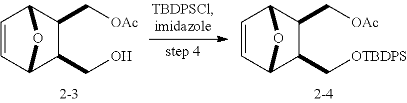

Imidazole (1.6 g, 23.7 mmol) and TBDPSCl (4.0 mL, 15.4 mmol) were added to a solution of 2-3 (2.5 g, 9.7 mmol) in dry DMF (20 mL) at ice bath under N₂ respectively. The reaction mixture was stirred at room temperature for 16 h, diluted with EtOAc (40 mL), washed with H₂O (20 mL*2) and sat. NaCl_{(aq)} (10 mL), dried out Na₂SO₄, filtered, and concentrated to yield crude 2-4 (4.9 g); R_{f}=0.52 (EtOAc/hexane 1:3).

### Step 5 Preparation of Compound 2-5

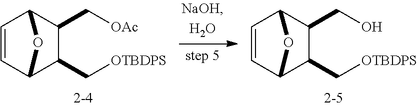

NaOMe (107 mg, 1.97 mmol) was added to a stirred solution of crude silyl ether 2-4 (4.9 g, 9.9 mmol) in MeOH (40 mL). The mixture was stirred at room temperature for 2 h, diluted with sat. NaCl_{(aq)} (40 mL), and extracted with EtOAc (40 mL*3). The combined extracts were dried out Na₂SO₄, filtered and concentrated. The residue was purified by column chromatography on silica gel (EtOAc/hexane 1:3, R_{f}=0.25) to provide 3.7 g of 2-5 (8.2 mmol, 83%).

### Step 6 Preparation of Compound 2-6

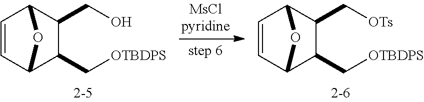

Et₃N (1.0 mL, 7.3 mmol), TsCl (0.69 g, 3.6 mmol), and 4-DMAP (44 mg, 0.36 mmol) were added to a solution of 2-6 (1.1 g, 2.4 mmol) in dry CH₂Cl₂ (15 mL) at ice bath under N₂. The reaction mixture was stirred at room temperature for 16 h, diluted with CH₂Cl₂ (10 mL), washed with H₂O (10 mL*2), sat. NaCl_{(aq)} (10 mL), dried out Na₂SO₄, filtered and concentrated. The residue was purified by column chromatography on silica gel (EtOAc/hexane 1:5, then EtOAc/hexane 1:3, R_{f}=0.5) to provide 1.3 g of 2-6 (2.1 mmol, 88%).

### Step 7 Preparation of Compound 2-7

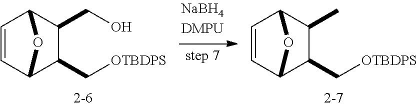

NaBH₄ (398 mg, 10.5 mmol) was added to a solution of 2-6 (1.28 g) in DMPU (6.5 mL) at ice bath. The reaction mixture was heated at 90-100° C. oil bath for 2 h, cooled in ice bath, quenched with H₂O, then stirred for 1 h, extracted with EtOAc (20 mL*2). The combined organic layer was washed with H₂O (10 mL*2) and sat NaCl_{(aq)} (5 mL), dried out Na₂SO₄, filtered and concentrated. The residue was purified by column chromatography on silica gel (EtOAc/hexane 1:10, R_{f}=0.26, then 1:3) to provide 625 mg of 2-7 (1.42 mmol, 69%)

### Step 8 Preparation of Compound 2-8

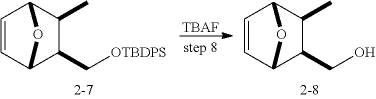

n-Bu₄NF (1.0 M solution in THF, 1.6 mL, 1.6 mmol) was added to a solution of 2-7 (580 mg, 1.32 mmol) in THF (13 mL). The reaction mixture was stirred for 16 h, removed the solvent, The residue was purified by column chromatography on silica gel (EtOAc:hexane, 1:1, R_{f}=0.25) to provide 255 mg of 2-8 (1.23 mmol, 97%)

### Step 9 Preparation of Compound 2-9

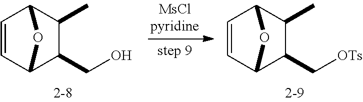

To a solution of Compound 2-8 (8.3 g, 59 mmol) in CH₂Cl₂ (210 mL) at ice bath were added Et₃N (21.0 mL, 148 mmol), 4-DMAP (1.0 g, 8.9 mmol), and TsCl (16.9 g, 88.8 mmol). The mixture was allowed to warm to room temperature and stirred for 16 h, washed with H₂O (100 mL×3) and brine (100 mL). The organic layer was dried over Na₂SO₄ and concentrated in vacuo. The residue was purified by column chromatography on silica gel (EtOAc:hexane, 1:3, R_{f} 0.46) to provide 14.8 g (50.2 mmol, 85%) of 2-9 as a colorless oil. EI-MS, m/z 317 [M+Na]⁺; [α]²⁴_{D}−14.8 (c 2.34, CHCl₃); ¹H (600 MHz; CDCl₃) δ 1.04 (3H, d, J=7.3 Hz), 1.83-1.90 (1H, m), 1.91-1.97 (1H, m), 2.51 (3H, s), 4.02 (1H, t, J=9.8 Hz), 4.22 (1H, dd, J=9.5 and 5.4 Hz), 4.49 (1H, s), 4.75 (1H, s), 6.32 (1H, dd, J=5.8 Hz and 1.6 Hz), 6.41 (1H, dd, J=5.8 and 1.6 Hz), 7.43 (2H, d, J=8.2 Hz), 7.87 (2H, d, J=8.2 Hz); ¹³C (150 MHz; CDCl₃) δ 14.1, 21.4, 33.6, 39.2, 71.0, 80.0, 84.5, 127.6, 129.7, 132.6, 134.4, 135.9, 144.7.

### Step 10 Preparation of Compound 2-10

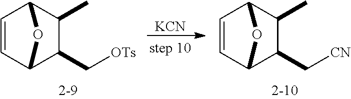

Compound 2-10 was prepared by reaction of tosylate 2-9 with KCN followed the known procedure.

### Step 11 Preparation of Compound 2-11

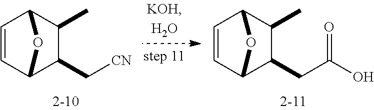

The nitrile 2-10 (6.7 g, 45 mmol) was refluxed for 4 h in 1N potassium hydroxide solution (480 mL, 480 mmol). After 4 h, the mixture was concentrated. The residue was allowed to cool to ice bath, acidified to pH 1 with conc. HCl_{(aq)}, and extracted with EtOAc (300 mL×3). The combined organic fractions were dried over Na₂SO₄ and concentrated in vacuo to yield acid (7.4 g, 44 mmol, 98%). TLC R_{f} 0.63 (EtOAc:hexane, 2:1); EI-MS, m/z 191 [M+Na]⁺; [α]²⁴_{D}−7.03 (c 1.95, CHCl₃); ¹H (600 MHz; CDCl₃) δ 1.00 (3H, d, J=7.3 Hz), 1.77-1.84 (1H, m), 1.98-2.04 (1H, m), 2.39 (1H, dd, J=16.9 and 10.0 Hz), 2.51 (1H, dd, J=16.9 and 5.4 Hz), 4.45 (1H, s), 4.65 (1H, s), 6.31 (2H, s); ¹³C (150 MHz; CDCl₃) δ 15.3, 33.5, 34.0, 35.9, 82.8, 84.8, 135.1, 135.6, 179.2.

### Step 12 Preparation of Compound 2-12

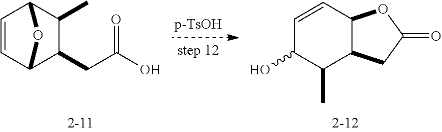

The acid 2-11 (4.4 g, 26.1 mmol) and p-TsOH (992 mg, 5.22 mmol) were in 20 mL of H₂O, refluxed overnight. The mixture was extracted with EA (20 mL*2), dried out Na₂SO₄, filtered and concentrated. The residue was purified by column chromatography on silica gel (EtOAc/hexane 2:1, R_{f}=0.43, then EA) to yield 3.0 g of 2-12 (17.8 mmol, 68%).

### Example 3

### Preparation of an Exemplary Compound 36a from Lactone 35a

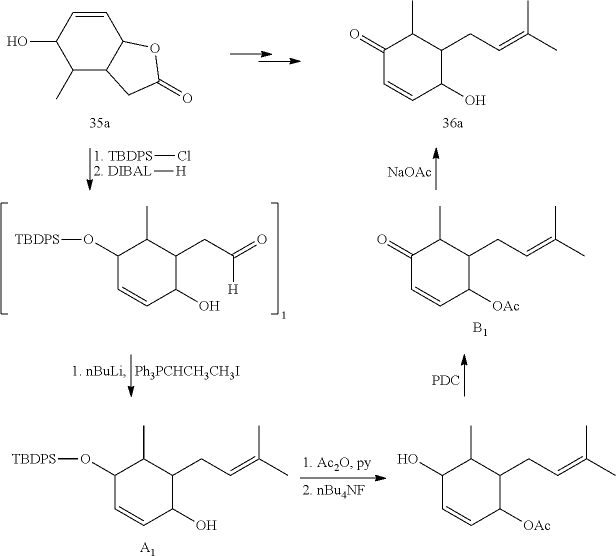

Compound 36a was prepared from Compound 35a under the following steps.

### Step 13 Preparation of Compound 2-13

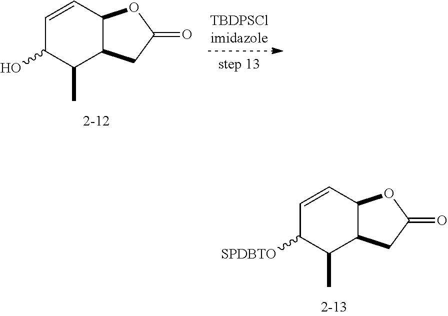

Imidazole (2.43 g, 35.7 mmol) and TBDPSCl (7.0 mL, 26.8 mmol) were added to a solution of 2-12 (3.0 g, 17.8 mmol) in dry DMF (30 mL) at ice bath under N₂ respectively. The reaction mixture was stirred at room temperature for 16 h, diluted with EtOAc (50 mL), washed with H₂O (20 mL*2) and sat. NaCl_{(aq)} (10 mL), dried out Na₂SO₄, filtered, and concentrated. The residue was purified by column chromatography on silica gel (EtOAc/hexane 1:5) to yield 5.7 g of 2-13 (14.0 mmol, 78%); R_{f}=0.55 (EtOAc/hexane 1:3).

### Step 14 Preparation of Compound 2-14

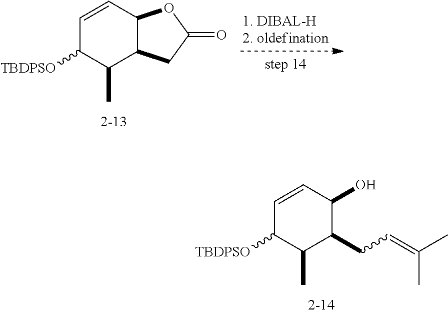

DIBAL-H (11.0 mL, 11.0 mmol) was dropped to a solution of 2-13 (2.2 g, 5.4 mmol) in dry DCM (10 mL) at −78 OC under N₂. The mixture was stirred for 1 h. The reaction was quenched by sat. NH₄Cl_{(aq)} (4 mL), stirred at rt for 1 h, filtered and concentrated to give crude the hemiacetal 2.28 g. Dry THF was dropped to the mixture of KOtBu (1.4 g, 12.5 mmol) and phosphonium salt (5.6 g, 13.0 mmol) in ice bath under N₂ to form ylide. After 10 min, the solution of hemiacetal was dropped to the solution of ylide. The mixture was refluxed for 2 h, quenched with sat. NH₄Cl_{(aq)} (10 mL), extracted with EA (20 mL*2), washed with sat. NaCl_{(aq)} (10 mL), dried out Na₂SO₄, filtered and concentrated. The residue was purified by column chromatography on silica gel (EtOAc/hexane 1:10 then 1:5) to yield 1.9 g of 2-14 (4.3 mmol, 80%); R_{f}=0.56 (EtOAc/hexane 1:3).

¹H (600 MHz; CD₃Cl) δ 0.77 (3H, d, J=7.3 Hz), 1.07 (9H, s), 1.65 (3H, s), 1.73 (3H, s), 1.84-1.90 (1H, m), 2.02-2.10 (1H, m), 2.21-2.28 (2H, m), 3.93 (1H, t, J=4.0 Hz), 4.18 (1H, br), 5.20-5.25 (1H, m), 5.66 (1H, dd, J=9.9 Hz and 4.5 Hz), 5.85 (11H, dd, J=9.9 Hz and 4.0 Hz), 7.37-7.41 (4H, m), 7.42-7.46 (2H, m), 7.66-7.70 (4H, m).

### Step 15 Preparation of Compound 2-15

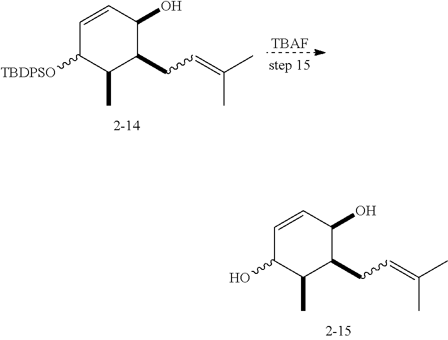

n-Bu₄NF (1.0 M solution in THF, 1.1 mL, 1.1 mmol) was added to a solution of 2-14 (400 mg, 0.92 mmol) in THF (7 mL). The reaction mixture was stirred for 16 h, removed the solvent, The residue was purified by column chromatography on silica gel (EtOAc/hexane, 1:1, R_{f}=0.48) to provide 178 mg of 2-15 (0.91 mmol, 99%).

### Step 16 Preparation of Compounds 2-16a, 2-16b, and 2-16c

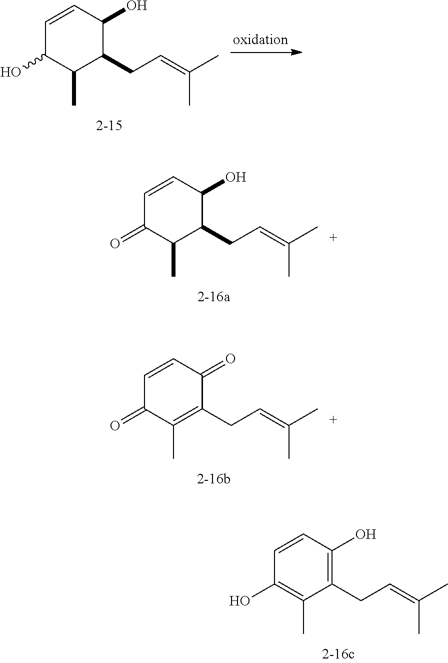

PDC (127 mg, 0.34 mmol) and trace 4 Å molecular sieve were added to the solution of 2-15 (80 mg, 0.41 mmol) in CH₂Cl₂ (8 mL). The mixture was stirred at room temperature overnight, diluted with ether (8 mL), and filtered. The residue was concentrated in vacuum and purified by column chromatography on silica gel (EtOAc/hexane, 1:5 then 1:2, R_{f}=0.31) to provide 19 mg of 2-16a (0.098 mmol, 24%); 2-16b (5 mg, 0.026 mmol, 6.3%, R_{f}=0.42, EtOAc/hexane, 1:5); 2-16c (4 mg, 0.020 mmol, 4.9%, R_{f}=0.44, EtOAc/hexane, 1:2).

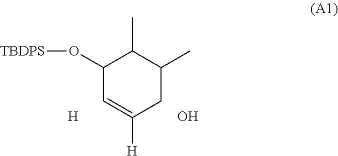

Exemplary Compounds 1 and 3 were prepared via Examples 1-3.

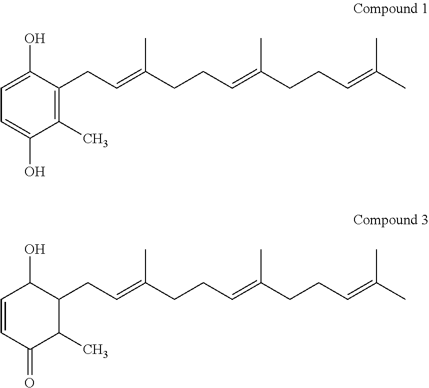 [SMILES: CC(C)=CCC/C(C)=C/CC/C(C)=C/Cc1c(O)ccc(O)c1C ; CC(C)=CCC/C(C)=C/CC/C(C)=C/CC1C(O)C=CC(=O)C1C]

The following compounds are prepared accordingly.

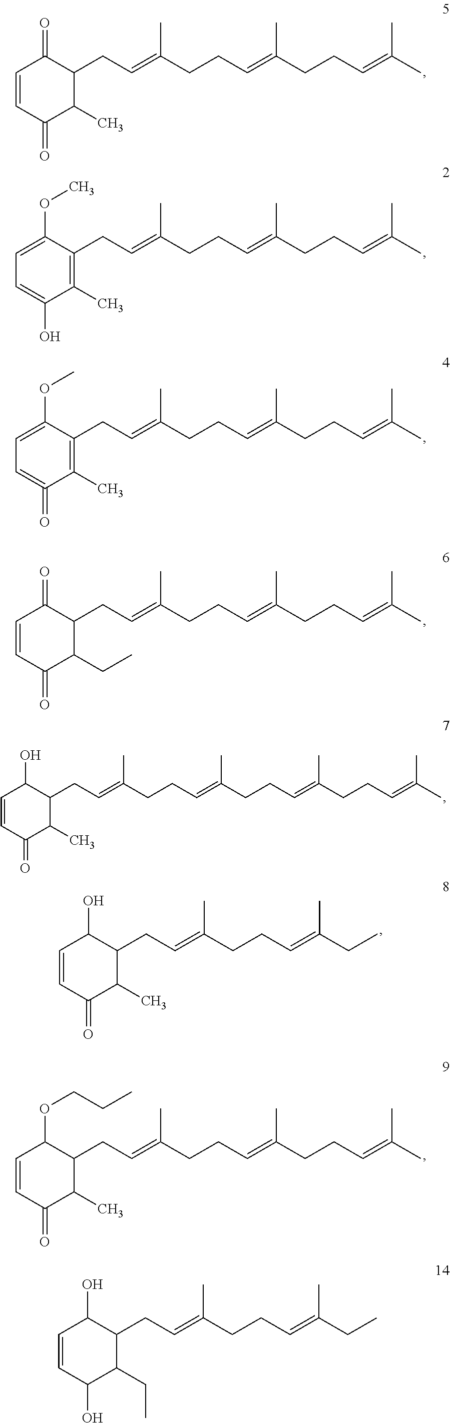

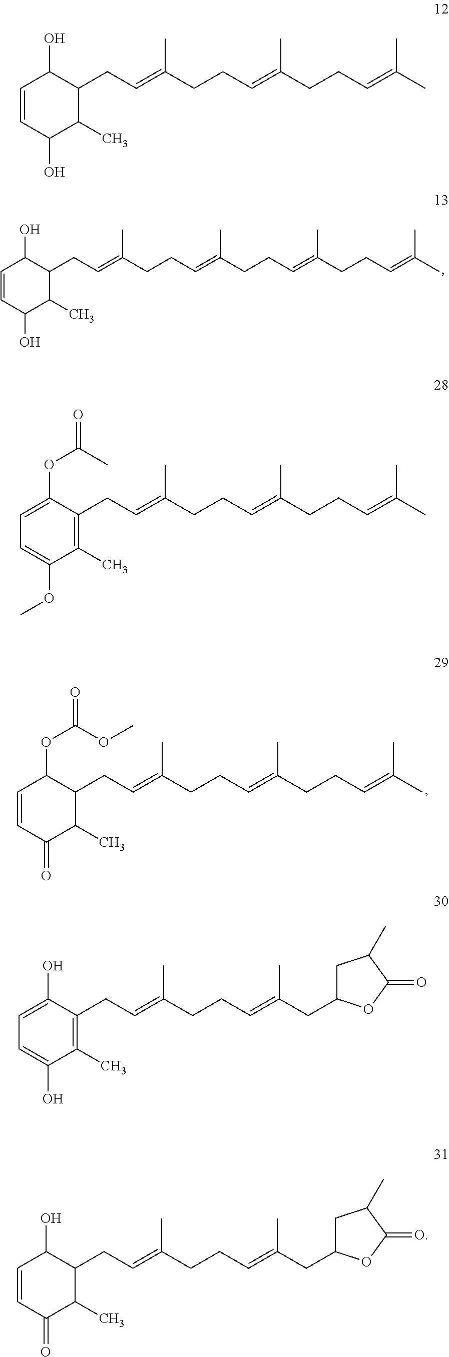 [SMILES: CC(C)=CCC/C(C)=C/CC/C(C)=C/CC1C(O)C=CC(O)C1C ; CC(C)=CCC/C(C)=C/CC/C(C)=C/CC/C(C)=C/CC1C(O)C=CC(O)C1C ; COc1ccc(OC(C)=O)c(C/C=C(\C)CC/C=C(\C)CCC=C(C)C)c1C ; COC(=O)OC1C=CC(=O)C(C)C1C/C=C(\C)CC/C=C(\C)CCC=C(C)C ; C/C(=C\Cc1c(O)ccc(O)c1C)CC/C=C(\C)CC1CC(C)C(=O)O1 ; C/C(=C\CC1C(O)C=CC(=O)C1C)CC/C=C(\C)CC1CC(C)C(=O)O1]

### Example 5

### Determining the Cytotoxic Effects of Exemplary Anticancer Agents

Human hepatoma (HepG2 and Hep 3B) and human breast cancer (MCF-7) cell lines were obtained from American Type Culture Collection (Rockville, Md., USA). HepG2 and Hep 3B cells were cultured in MEM alpha medium (Invitrogen/Gibco BRL, Grand Island, N.Y., USA) and MCF-7 cells were cultured in DMEM medium (Invitrogen/Gibco BRL). All cells were cultured at 37° C. in 5% CO₂ in culture media supplemented with 10% fetal bovine serum (Invitrogen/Gibco BRL) and 100 U/ml streptomycin and penicillin (Invitrogen/Gibco BRL). For treatment, cells were seeded in six-well plates at 6.25×10⁵ cells/well. On the following day, the media were changed to serum-free and the cells were serum-starved for 24 h. The test compounds were dissolved in DMSO separately and diluted to the required concentration with serum-free medium. Cultures were then treated with diluted test compounds for 1 h. After treatment, cells were washed with cold phosphate-buffered saline and lysed using RIPA lysis buffer containing phosphatase and protease inhibitors.

### MTT Assay

The MTT (3-(4, 5-dimethylthiazol-2-yl)-2,5-diphenyl tetrazolium bromide) cell viability assay is a colorimetric assay system, which measures the reduction of a tetrazolium component (MTT) into an insoluble blue/purple colored formazan product by succinate tetrazolium reductase in mitochondria of viable cells. The absorbance of the complex is read spectrophotometrically and is directly proportional to the number of live or viable cells. Formazan formation can therefore be used to assess and determine the survival rate of cells.

Cancer cells were suspended in 10% fetal bovine serum (Life Technologies Inc.) containing F-12K culture medium that also includes 1% penicillin and 1% streptomycin. Cells were cultured under 5% CO2, 37° C. and 95% humidity. After cell proliferation, the cells were washed once with PBS, treated with the trypsin-EDTA, and then centrifuged at 1,200 rpm for 5 minutes to separate cells from supernatant. The cells were re-suspended in fresh culture medium (10 ml) and placed in 96 well plates.

To each of the 96 well plates seeded at a density of 5,000 cells per well, a known concentration of test compounds were added individually. The 96 well plates were incubated at 37° C., 5% CO₂ for 48 hours. Subsequently, in the dark environment to each well of the plates were added 2.5 mg/ml of MTT. The reaction was subsequently terminated by addition of 100 μl of lysis buffer after 4 hours. The survival rate of cells was calculated based on the measurement of absorption at the 570 nm wavelength by enzyme immunoassay analyzer. The IC50 value was determined by a nonlinear curve fitting program using the GraphPad prism software v 4.01.

TABLE 1
IC₅₀ values of exemplary compounds determined by MTT assay.
Suprisingly, these cyclohexenone compounds show an unexpected superior
inhibition against the test cancer cells compared to their known analogs,
such as 4-hydroxy-2,3-dimethoxy-6-methyl-5-(3,7,11-trimethyldodeca-
2,6,10-trienyl)cyclohex-2-enone that has IC₅₀ of >30 μM against
Hep 3B (based on the prior published result).

| Compound | Hep3B | HepG2 | MCF-7 |
|---|---|---|---|
| 1 | 0.3254 | 0.9484 | 0.5193 |
| 3 | 0.0043 | 0.1555 | 0.1075 |

All IC50 Values were the Average of at Least Six Independent Experiments.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of formula II: 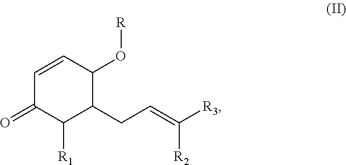
or a pharmaceutically acceptable salt, metabolite, solvate or prodrug thereof, wherein R is a hydrogen, C(═O)OR₅, C(═O)R₅, C(═O)NR₅R₆, or a C₁-C₁₂alkyl, R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

2. A compound of formula V: 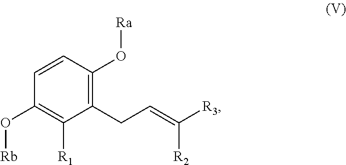
or a pharmaceutically acceptable salt, metabolite, solvate or prodrug thereof, wherein each of Ra and Rb is a hydrogen, C(═O)OR₅, C(═O)R₅, C(═O)NR₅R₆, or a C₁-C₁₂alkyl
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

3. The compound of claim 1, wherein R is a hydrogen, C(═O)C₃H₈, C(═O)C₂H₅, or C(═O)CH₃.

4. The compound of claim 1, wherein each of R₁, R₂ and R₃ independently is hydrogen, methyl, ethyl, propyl, butyl, pentyl, hexyl, or heptyl.

5. The compound of claim 1, wherein each of R₂ and R₃ independently is (CH₂CH═C(CH₃)(CH₂))ₘ—R₄.

6. The compound of claim 5, wherein R₄ is H, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂.

7. The compound of claim 5, wherein R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

8. The compound of claim 7, wherein R₄ is C₁—C alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

9. The compound of claim 8, wherein R₄ is CH₂CH═C(CH₃)₂.

10. The compound of claim 1, wherein said compound is

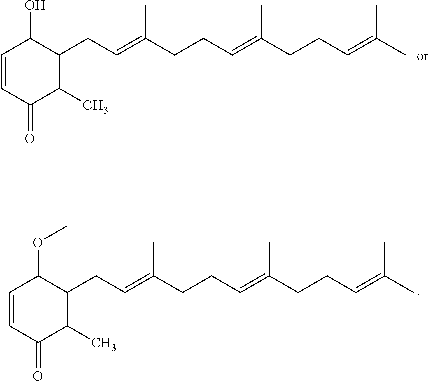 [SMILES: CC(C)=CCC/C(C)=C/CC/C(C)=C/CC1C(O)C=CC(=O)C1C ; COC1C=CC(=O)C(C)C1C/C=C(\C)CC/C=C(\C)CCC=C(C)C]

11. The compound of claim 2, wherein R is a hydrogen, C(═O)C₃H₈, C(═O)C₂H₅, or C(═O)CH₃.

12. The compound of claim 2, wherein each of R₁, R₂ and R₃ independently is hydrogen, methyl, ethyl, propyl, butyl, pentyl, hexyl, or heptyl.

13. The compound of claim 2, wherein each of R₂ and R₃ independently is (CH₂CH═C(CH₃)(CH₂))ₘ—R₄.

14. The compound of claim 2, wherein said compound is

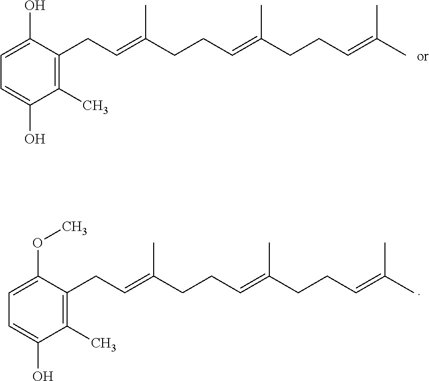 [SMILES: CC(C)=CCC/C(C)=C/CC/C(C)=C/Cc1c(O)ccc(O)c1C ; COc1ccc(O)c(C)c1C/C=C(\C)CC/C=C(\C)CCC=C(C)C]

15. A processes for preparing a compound of formula VI: 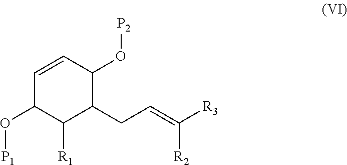
comprising a step of reacting a compound of formula II, 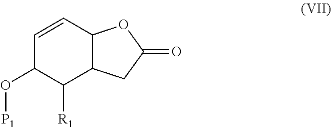
(VII) with a compound (VIII), Ph₃PCHR₂R₃L (VIII), in the presence of a reducing agent, and a base,
wherein L is a leaving group, each of P₁ and P₂ is a hydroxyl protecting group or R;
R is a hydrogen, C(═O)OR₅, C(═O)R₅, C(═O)NR₅R₆, or a C₁-C₁₂alkyl;
R₁ is C₁-C₁₂alkyl, NR₅R₆, OR₅, SR₅, or halogen;
each of R₂ and R₃ independently is a hydrogen, an optionally substituted C₁-C₁₂alkyl or (CH₂CH═C(CH₃)(CH₂))ₘ—R₄, wherein
R₄ is H, NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently H or C₁-C₈alkyl;
R₇ is a C₁-C₈alkyl, OR₅ or NR₅R₆;
m=0-11.

16. The process of claim 15, wherein each of R₁, R₂ and R₃ independently is H, methyl, ethyl, propyl, butyl, pentyl or hexyl.

17. The process of claim 15, wherein each of R₂ and R₃ independently is (CH₂CH═C(CH₃)(CH₂))ₘ—R₄.

18. The process of claim 17, wherein R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

19. The process of claim 15, wherein said base is a lithium salt.

20. The process of claim 19, wherein said lithium salt is n-butyllithium.
